(19) United States
(12) Patent Application Publication
(10) Pub. No.: US 2005/0013882 A1
(43) Pub. Date: Jan. 20, 2005

(54) Phyto fluid

(76) Inventors: Jacques Owen, Danville (CA); Guido Di Lenardo, St. Leonard (CA)

Correspondence Address: James A. Sheridan;Pandiscio & Pandiscio, P.C., 470 Totten Pond Road, Waltham, MA, 02451-1914, US

(21) Appl. No.: 10/826,829

(22) Filed: Apr. 16, 2004

Related U.S. Application Data
(60) Provisional application No. 60/463,029, filed on Apr. 16, 2003.

(51) Int. Cl. A61K 35/78

(52) U.S. Cl. 424/742; 424/745; 424/770

(57) ABSTRACT

The present invention relates to a novel plant-derived fluid, or phyto fluid, having demonstrated germicidal (anti-bacterial and anti-mould) properties. The present invention further relates to uses for this fluid. Advantageously, this phyto fluid is self-atomizing and does not require direct contact with surfaces to eliminate bacteria and mould. This phyto fluid may be used in homes, hospitals, office buildings, airplanes and other public areas to sanitize bacteria and mould.

### REFERENCE TO PENDING PRIOR PATENT APPLICATION

This patent application claims benefit of pending prior U.S. Provisional Patent Application Ser. No. 60/463,029, filed Apr. 16, 2003 by Jacques Owen for PHYTO FLUID, which patent application is hereby incorporated herein by reference.

### FIELD OF THE INVENTION

The present invention relates to a novel plant-derived fluid, or phyto fluid, having demonstrated germicidal (anti-bacterial and anti-mould) properties and to uses for this fluid. Advantageously, this phyto fluid is self-atomizing and does not require direct contact with surfaces to eliminate bacteria and mould. This phyto fluid may be used in homes, hospitals, office buildings, airplanes and other public areas to sanitize bacteria and mould.

### BACKGROUND OF THE INVENTION

Essential oils (also known under a number of alternative names, such as ethereal or plant oils), are complex mixtures of chemical compounds (typically, aldehydes, phenols, terpene hydrocarbons, ketones, esters) whose compositions vary from species to species, and even between parts of the same plant. Some of these compounds, in particular phenols, are known to possess strong germicidal activity.

Isolated from plants or various parts thereof by a variety of techniques, including solvent extraction and steam distillation, essential oils may be further purified and fractionated. In the pure state, some components may be low-melting solids, but even these components are usually volatile enough to evaporate (sublimate) at room temperature. The same may also be said about their liquid components, some of which may be relatively high-boiling liquids.

Essential oils as mixtures of their various components, including the solid ones, are normally liquid at room temperature. The history of essential oils is almost as old as the history of mankind itself, even though for much of this time they were mostly appreciated for their fragrant rather than therapeutic properties. Sample patents relating to the use of various essential oils, mixtures or components thereof for aerial disinfection of closed spaces, or as nasal/pulmonary antiseptics include the following: U.S. Pat. No. 6,142,383, U.S. Pat. No. 6,086,904, U.S. Pat. No. 5,635,132, U.S. Pat. No. 2,550,263 and U.S. Pat. No. 2,546,895. Sample patents disclosing compositions comprising essential oils derived from a variety of plants, such as thyme, lavender and eucalyptus for a variety of applications (which may include germicidal activity), are the following: U.S. Pat. No. 6,528,081, U.S. Pat. No. 6,514,541, U.S. Pat. No. 6,514,539, U.S. Pat. No. 6,447,816, U.S. Pat. No. 6,413,555 and U.S. Pat. No. 6,361,785. Examples of patents covering compositions based on essential oils for surface cleaning and disinfection are the following: U.S. Pat. No. 6,436,342, U.S. Pat. No. 6,346,281 and U.S. Pat. No. 6,165,964.

Despite the above, there appears to be a need (or market demand) for new formulations of essential oils that are suitable for the purposes described above.

### OBJECTS OF THE INVENTION

The general object of the present invention is therefore to provide a new phyto fluid that has germicidal properties and that is suitable for eliminating bacteria and mould in buildings, airplanes and other enclosed spaces without requiring direct contact with surfaces.

### SUMMARY OF THE INVENTION

In accordance with the present invention, there is provided a novel plant-derived fluid, or phyto fluid. The present invention further relates to uses for this fluid. This fluid has germicidal properties and a high volatility factor that make it suitable for the elimination of bacteria and mould in homes, office buildings, airplanes and other enclosed spaces without having to apply the fluid directly on the contact surfaces. This fluid may also be used as a liquid sanitizer to remove bacteria and mould on surfaces by direct application.

The phyto fluid is a liquid which evaporates at room temperature and whose vapour acts as the actual germicide with respect to either airborne or surface-deposited germs. The vapour also penetrates porous surfaces and mould spores which has the effect of extending the germicidal efficacy.

Other objects, advantages and features of the present invention will become more apparent upon reading of the following non restrictive description of preferred embodiments thereof, given by way of example only with reference to the accompanying drawings.

### DESCRIPTION OF THE PREFERRED EMBODIMENTS

Definitions: Unless otherwise specified, the terms used in the present application have the meanings that a person of skill in the art would normally attribute to them.

The term “fluid” is intended to refer to the novel plant-derived composition that is the subject of the present application. Specifically, this composition may be either in a liquid or gaseous state.

The term “bacteria” refers to extremely small, unicellular microorganisms that multiply by cell division and whose cell is typically contained within a cell wall, occurring in spherical, rod-like, spiral, or curving shapes and found in virtually all environments; some types are important agents in the cycles of nitrogen, carbon, and other matter, while others cause diseases in humans and animals.

The terms “mould” and “moulds” are defined as microscopic, plant-like organisms, composed of long filaments called hyphae. Mould hyphae grow over the surface and inside nearly all substances of plant or animal origin. Because of their filamentous construction and consistent lack of chlorophyll they are considered by most biologists to be separate from the plant kingdom and members of the kingdom of fungi. They are related to the familiar mushrooms and toadstools, differing only in not having their filaments united in large fruiting structures.

### DETAILED DESCRIPTION

The present invention relates to a novel fluid that is a 100% plant-based (phyto fluid) having germicidal properties. It is self atomizing in the air and does not require direct liquid contact to be effective. It is a fluid that is useful for eradicating or sanitizing bacteria and mould from, among other things, enclosed areas such as homes, hospitals, office buildings, airplanes and other public areas.

In chemical terms, the novel phyto fluid is comprised of a mixture of essential oils derived from the following natural sources or plant species: balsam fir, thyme, lavender, thuja and eucalyptus. The essential oils derived from each of these five sources are present in approximately equal volumes in the phyto fluid (i.e., the phyto fluid is comprised of approximately 20% essential oils derived from balsam fir, approximately 20% essential oils derived from thyme, approximately 20% essential oils derived from lavender, approximately 20% essential oils derived from thuja and approximately 20% essential oils derived from eucalyptus). Suitable sources include essential oils derived from the plants shown in Table 1.

TABLE 1
Plant Sources for Phyto Fluid

| Plant Source | Genus |
|---|---|
| Balsam fir | Abies (ex: A. balsamea, A. alba) |
| Thyme | Thymus (ex: T. vulgaris, T. capitans, |
| | T. camphoratus, T. mastichina, |
| | T. algeriensis, T. serpyllum) |
| Lavender | Lavandula (ex: L. vera, L. spica, |
| | L. officinalis, L. delphinensis, L. fragrans) |
| Thuja | Thuja (ex: T. occidentalis, T. plicata, |
| | T. articulata) |
| Eucalyptus | Eucalyptus (ex: E. globulus, |
| | E. radiata, E. rupicola, E. fastigiata, |
| | E. amygdalina, E. australiana, E. smithii, |
| | E. polybracta, E. piperata, |
| | E. polybracta, E. dives) |

The physical and chemical characteristics of the phyto fluid are indicated in Table 2.

TABLE 2
Physical and Chemical Characteristics of the Phyto Fluid

| Appearance at | |
|---|---|
| Room Temperature | Transparent liquid |
| Colour | Light yellow |
| Odour | Camphorated |
| Solubility | Soluble in oil, not very soluble in |
| | alcohol, not soluble in water |
| Density | 0.882-0.912 at 25° C. |
| Flash Point | 53° C. |
| % Volatility | 95% |

This formulation has a “balsamic” calming effect on bronchial trachea secretions (mucus) and is an overall anti-bacterial agent for the body. As an antibacterial vapour in the air it is an effective pulmonary antiseptic.

In one embodiment, a few drops of the novel phyto fluid are placed in a room or space and allowed to evaporate. In this way, the phyto fluid effectively eliminates bacteria and moulds in the air, and by settling on surrounding surfaces, on these surfaces as well.

In another embodiment, the novel phyto fluid, which may be ingested by animals, including humans, can be used to eliminate ear infections and to reduce pulmonary bacteria load. The formulation of this phyto fluid results in a volatile gas at normal room temperature (18-25° C.). Consequently, it reduces the bacteria load in the air and on surfaces. With time (the passage of a few hours), it will also penetrate porous surfaces and mould spores.

Advantageously, the air in the areas treated remains bacteria or mould free for an extended time (i.e., for at least three months).

The novel phyto fluid may be used in all environmental air clean operations by reducing bacteria and moulds in the air. Additionally, it may potentially be used in certain dentistry applications, such as to disinfect the mouth during dental interventions and to clear airborne bacteria in the dentist's office.

The phyto fluid has been used to treat pneumonia in veterinary applications with successful results.

With some modification, the phyto fluid may also be useful in sanitizing water to render it bacteria and germ free without the use of chlorine.

Uses or Applications for the Phyto Fluid

Among numerous applications possible, the phyto fluid described herein is expected to be effective for the following purposes:
1. As an air antiseptic for households with a mould or mildew load, which can contribute to asthma and other breathing ailments;
2. To reduce the mould and/or bacteria load in buildings such as schools, libraries, museums, hospitals, office buildings, warehouses, etc.;
3. To disinfect airplanes after flights;
4. For certain medical applications, such as treating ear infections, reducing pulmonary bacteria load and treating skin ailments; and
5. As a means of civil protection for effective biological contamination countermeasures of large public spaces such as airports, train and subway stations, concert halls and stadiums.

### EXAMPLE 1

### Efficiency of Phyto Fluid in Eliminating Bacteria and Mould

Disk Diffusion on Agar Medium

To test the efficacy of the phyto fluid on bacteria and mould, various species of bacteria and mould were grown on agar plates. A disk containing the phyto fluid was positioned in the middle of each agar plate, and the hallow surrounding the disk measured. The greater the diameter of the halo surrounding the disk, the more efficacious the phyto fluid proved to be against the microorganism tested. The disk had a diameter of 6 mm. A halo of 15 mm in diameter is considered to be indicative of a respectable activity.

The microorganisms tested and the results of the disk diffusion experiments are shown in Table 3. As may be seen from these results, the phyto fluid has a demonstrable germicidal activity.

TABLE 3
Results of Disk Diffusion Experiments

| | *Diameter of Halo Surrounding |
|---|---|
| Microorganism | Disk (mm) |
| Saccharomyces cerevisiae | 40-23-44 |
| Salmonella enteritidis | 38-47 |
| Trichoderma viride | 14-60-28 |
| Citrobacter freundii | 25-44-28 |
| Aspergillus niger | 17-46 |
| Candida albicans | 50-46-52-41-29-10-40-22-18 |
| Streptococcus faecalis | 26 |
| Staphylococcus aureus | 24-28-40-46-50-40-38-48 |
| Penicillium | 60-12 |
| Salmonella arizonae | 47-50-20-18-19-19-18 |
| Streptococcus mitis | 36-50-27-28-27-27 |
| Escherichia coli | 28-10 |
| Rhodotorula | 26-14 |
| Lactobacillus | 30-40 |
| Negative control (disk without phyto | 6 |
| fluid) | |

*Each number indicates the result of an experiment conducted against the microorganism indicated.
Up to nine (9) separate experiments were conducted with each microorganism.

### EXAMPLE 2

### Use of Phyto Fluid as a Household Sanitizer

The experiment was conducted in the bathroom of a regular house. Mould (mildew) was recurrent in such areas as the ceiling and on the window ledges. These surfaces were cleaned using a rag soaked with the phyto fluid. No mould or mildew could be detected for at least three months following the experiment.

### EXAMPLE 3

### Use of Phyto Fluid in a Five-Story Building Comprising Offices, Shops and Restaurants

Preliminary analyses indicated that a portion of the ventilation system was contaminated with mould (mildew). Sample readings of the bacterial and mould counts are shown in Table 4.

TABLE 4
Bacterial and Mould Counts in Selected Locations of Five-Story
Building Before Phyto Fluid Treatment

| | Total Bacteria | Total Mould |
|---|---|---|
| Location | (C.F.U./25 cm²) | (C.F.U/25 cm²) |
| Office A (5^{th} floor) | 180,000 | 10,000+ |
| Office B (5^{th} floor) | 200,000 | 10,000+ |
| Office C (4^{th} floor) | 21,500 | 10,000+ |
| Office D (4^{th} floor) | 58,000 | 10,000+ |

The entire office building (having a surface area of approximately 11,500,000 square feet) was treated using 500 ml of the phyto fluid. Soaked “pads” were introduced into the ventilation system so as to distribute the fluid uniformly throughout the building. Prior to this exercise, the fresh air vent was closed and the heating system shut down so as to allow air recirculation and keep fluid vapours within the building.

Treatment occurred on a Friday evening after the close of business. The fresh air vent was reopened on the following Sunday afternoon. The scent generated by the phyto fluid was barely perceptible, and the cleaning staff did not notice it before being questioned about it.

Four days later, new air samples were taken to measure the bacteria and mould counts. The results are shown in Table 5.

TABLE 5
Bacterial and Mould Counts in Selected Locations of Five-Story
Building After Phyto Fluid Treatment

| | Total Bacteria | Total Mould |
|---|---|---|
| Location | (C.F.U/25 cm²) | (C.F.U/25 cm²) |
| Office A (5^{th} floor) | 30 | 110+ |
| Office B (5^{th} floor) | 10 | less than 10 |
| Office C (4^{th} floor) | less than 10 | less than 10 |
| Office D (4^{th} floor) | 30 | 65 |

It should be noted that the areas where a small quantity of mould remained were areas where the phyto fluid could not be introduced directly. These areas could only be treated by recirculated air. Nevertheless, the effectiveness of the phyto fluid in eliminating bacteria and mould is significant.

Although the present invention has been described hereinabove by way of preferred embodiments thereof, it can be modified without departing from the spirit, scope and nature of the subject invention, as defined in the appended claims.

1. A phyto fluid comprising essential oils from the following plant sources: balsam fir, thyme, lavender, thuja and eucalyptus.

2. A phyto fluid as defined in claim 1, wherein the essential oils from each of the plant sources is present in approximately equal volumes.

3. Use of a phyto fluid as defined in claim 1 to reduce bacteria and moulds in the air.

4. Use of a phyto fluid as defined in claim 1 as an air antiseptic for households with a mould or mildew loads.

5. Use of a phyto fluid as defined in claim 1 to reduce the mould or bacteria load in buildings such as schools, libraries, museums, hospitals, office buildings, warehouses, concert halls and stadiums.

6. Use of a phyto fluid as defined in claim 1 to disinfect airplanes after flights.

7. Use of a phyto fluid as defined in claim 1 as a means of civil protection for effective biological contamination countermeasures of large public spaces such as airports, train and subway stations, concert halls and stadiums.
